(19) United States
(12) Patent Application Publication
(10) Pub. No.: US 2016/0106872 A1
(43) Pub. Date: Apr. 21, 2016

(54) HANDHELD DEVICE FOR DESTROYING MICROORGANISMS

(71) Applicant: Seth Martinez, Chicago, IL (US)

(72) Inventor: Seth Martinez, Chicago, IL (US)

(21) Appl. No.: 14/610,816

(22) Filed: Jan. 30, 2015

Related U.S. Application Data
(60) Provisional application No. 62/065,136, filed on Oct. 17, 2014.

(51) Int. Cl. A61L 2/10

(52) U.S. Cl. CPC A61L 2/10

(57) ABSTRACT

A handheld sanitizing device with a housing having an internal passageway with an opening at a first end of the internal passageway and at least one radiation emitting device capable of emitting radiation that can destroy microorganisms at a second end of the internal passageway. The internal passageway includes a reflective material over at least a portion. Preferably the radiation emitting device comprises an LED capable of emitting UV-C light. In some embodiments, the device is a key fob sized device. The device may also be sized to house a cellular phone.

### CROSS-REFERENCES TO RELATED APPLICATIONS

This application claims priority to U.S. Provisional Application No. 62/065,136 filed on Oct. 17, 2014, the entirety of which is incorporated by reference.

### FIELD OF THE INVENTION

The present invention relates to a handheld device that can be used to destroy microorganisms on an object, and more particularly a handheld device that uses radiation, such as light, to destroy microorganisms on an object.

### BACKGROUND OF THE INVENTION

There are various methods and devices that are capable of being utilized to remove germs, bacteria and/or other microorganisms from such objects. For example, it is known to use liquids, such as alcohols, acids and bases, to clean hands or other objects and surfaces. However, it is believed that using liquids on objects, such as objects placed into a person's mouth, is undesirable because of the taste associated with same. Furthermore, some liquid-based cleaners or bactericides, like an acid or base, may be dangerous to ingest thereby making these liquids also undesirable to use with objects placed into a person's mouth. Additionally, some liquid-based cleaners may damage or destroy fabric, electrical equipment, or other materials and surfaces, making the cleaners unusable for those surfaces.

It is also known to use radiation, such as light, to clean objects by using the light to destroy microorganisms on the surface of the objects. For example, ultraviolet (“UV”) light with a wavelength between approximately 100 to 290 nanometers (also referred to as “UV-C light”) can be used to destroy the DNA in microorganisms and thereby destroy microorganisms. However, many of the devices that use UV-C light are large and bulky, making such devices difficult to move around and use with the ease of other bactericidal devices, like the liquid bactericidal discussed above. Moreover, some of the smaller UV-C light bactericidal devices are portable, but may require a wired connection to an electrical outlet or are too large to carry around inconspicuously.

Therefore, it would be desirable to have a device for destroying microorganisms that has the portability and size benefits of the liquid cleaners and is able to be used in a wide variety of applications like the light-based devices.

### SUMMARY OF THE INVENTION

A new handheld device has been invented which uses a radiation emitting element to destroy microorganisms like germs, bacteria, viruses, yeast, and mold.

In a first aspect of the present invention, the invention may be broadly characterized as providing a handheld bactericidal device comprising: a housing and at least one radiation emitting device. The housing includes an internal passageway with an opening at a first end and terminating at a second end within the housing. The at least one radiation emitting device is disposed within the internal passageway and is capable of emitting radiation that can destroy the DNA of microorganisms. An inner surface of the internal passageway comprises a reflective material.

In at least one embodiment of the present invention, the first end of the internal passageway is wider than the second end of the internal passageway.

In some embodiments of the present invention, all of the inner surface of the internal passageway comprises the reflective material. It is contemplated that the reflective material comprises polished aluminum.

In at least one embodiment of the present invention, the at least one radiation device comprises an LED capable of emitting UV-C light. It is contemplated that the handheld device further comprises at least three LEDs capable of emitting UV-C light.

In at least one embodiment of the present invention, the housing further comprises an extension extending away from the housing and an aperture disposed in the extension.

In at least one embodiment of the present invention, the housing includes a cavity and the cavity is configured to contain a cellular telephone.

In at least one embodiment of the present invention, the device further comprises a solar panel disposed in the housing.

In at least one embodiment of the present invention, the reflective material comprises polished aluminum.

In at least one embodiment of the present invention, the internal passageway comprises a smooth surface.

In at least one embodiment of the present invention, the internal passageway comprises an angular surface.

In at least one embodiment of the present invention, the first end of the internal passageway comprises a lens.

In some embodiments of the present inanition, the device comprises a key fob sized device.

In a second aspect of the present invention, the present invention provides a bactericidal device comprising a housing, at least one LED capable of emitting UV-C light, and a solar panel. The housing includes an internal passageway with an opening at a first end and terminating at a second end within the housing. The opening is disposed in a side surface of the housing. The at least one LED capable of emitting UV-C light is disposed within the internal passageway. The solar panel is disposed in the housing.

In at least one embodiment of the present invention, the opening comprises a lens.

In at least one embodiment of the present invention, the housing further comprises a cavity configured to receive a cellular telephone.

In at least one embodiment of the present invention, the housing further comprises an extension extending away from the housing and an aperture disposed in the extension.

In at least one embodiment of the present invention, the device further comprises an actuating element in the housing. It is contemplated that the actuating element comprises a button.

In at least one embodiment of the present invention, the device further comprises a power source disposed within the housing.

These embodiments, aspects and features of the invention are contemplated to be combined in any manner, as would be appreciated by those of skill in the art.

These and other embodiments and aspects of the present invention will be apparent to those of ordinary skill in the art.

### BRIEF DESCRIPTION OF THE DRAWINGS

The figures in the appended drawing will make it possible to understand how the invention can be produced and used. In these figures, identical reference numbers denote similar elements.

FIG. 1 is a top and front isometric exploded view of a device according to one or more embodiments of the present invention.

FIG. 2 is a bottom and front isometric view of the device according to one or more embodiments of the present invention shown in FIG. 1.

FIG. 3 is a top and front isometric view of the device according to one or more embodiments of the present invention shown in FIG. 1.

FIG. 4 is a top and front isometric exploded view of a device according to one or more embodiments of the present invention.

FIG. 5 is a bottom and front isometric view of the device according to one or more embodiments of the present invention shown in FIG. 4.

FIG. 6 is a top and front isometric view of the device according to one or more embodiments of the present invention shown in FIG. 4.

FIG. 7 is a top and front isometric view of a passageway that may be used in a device according to one or more embodiments of the present invention.

FIG. 8 is a front view of another passageway that may be used in a device according to one or more embodiments of the present invention.

### DETAILED DESCRIPTION OF THE PREFERRED EMBODIMENTS

A device according to one or more embodiments comprises a bactericidal device that is handheld and utilizes an emitted radiation, such as U-VC light, for destroying microorganisms. The device allows for the ease of portability typically associated with liquid cleaners but may be used in a wide range of applications associated like the light-based bactericidal devices, discussed above. Furthermore, in some embodiments the device includes a solar panel which allows the device to be charged when not in use.

With these broad principles in mind, one or more specific embodiments of the present invention will now be described with the understanding that these are merely exemplary embodiments and are not intended to be limiting.

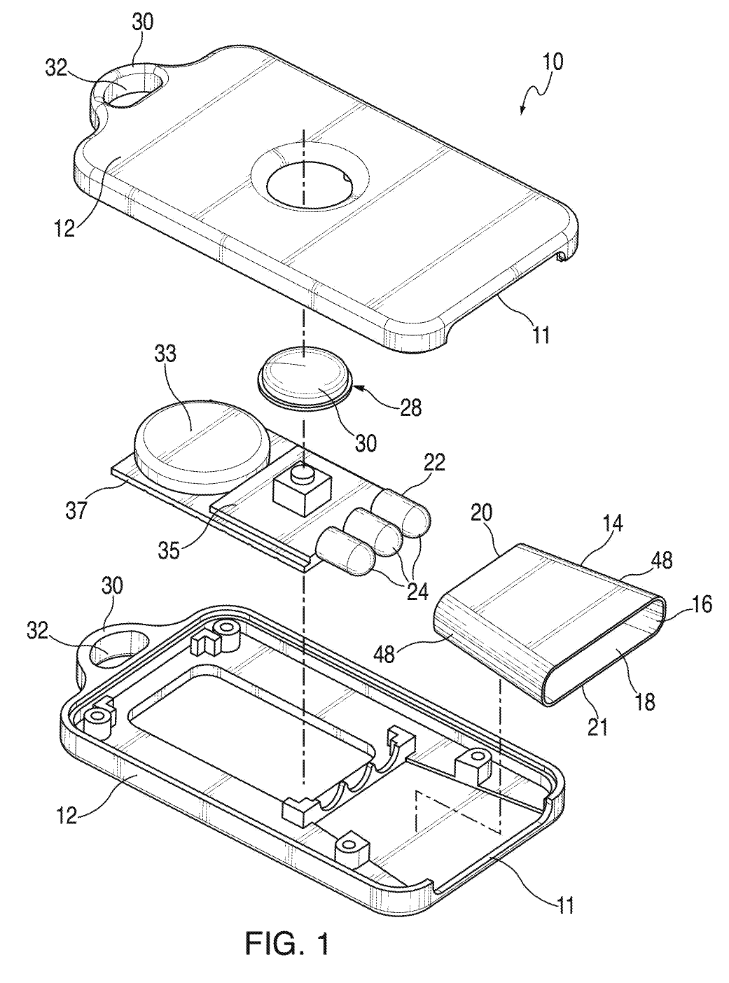

With reference to FIGS. 1 to 3, a handheld bactericidal device 10 broadly comprises a housing 12. As shown in FIG. 1, the housing 12 comprises a top 12a and a bottom 12b. The top 12a and the bottom 12b may be held together by external fasteners such as screws or the like, or by an internal interference fastening relationship. Other configurations may be used. An internal passageway 14 is disposed, at least partially, inside of the housing 12. A first end 16 of the passageway 14 includes an opening 18 disposed adjacent an opening 11 in the housing 12. A second end 20 of the passageway is disposed within the housing 12. As shown in FIG. 1, the first end 16 of the passageway 14 is wider than the second end 20 of the passageway 14.

At least one radiation emitting device 22 capable of emitting radiation that is capable of destroying microorganisms, or otherwise killing microorganisms, is disposed at the second end 20 of the passageway 14. It is preferred that the at least one radiation emitting device 22 comprises an LED 24 capable of emitting a UV-C light which is capable of destroying a microorganism. Preferably, more than one LED 24 is used, and most preferably at least three LEDs 24 are used. Furthermore, the radiation emitting devices 22 do not have to all be the same, for example, the device 10 may include at least one UV-C LED, at least one UV-B LED, and at least one UV-A LED. The UV-A and UV-B can be used to locate some microorganisms while the UV-C may then be used to destroy the microorganisms. Although the remaining description is in relation to the preferred embodiment with UV-C light emitted from an LED 24, other radiation emitting devices 22 may likewise be used in accordance with the principles of the present invention, such as UV-A LEDs and U-VB LEDs.

Returning to the Figures, the UV-C light travels from the LED 24, through the passageway 14, though the first end 16 of the passageway 14 and out of the opening 18 in the passageway 14 and the opening 11 in the housing 12. It is contemplated that the opening 11 in the housing or the opening 18 in the passageway 14 is adjustable in size utilized to allow for an adjustable beam to be utilized. For example, an adjustable blade aperture (like those used in a camera) may be utilized which will allow the size of the opening 11 in the housing 12 or the opening 18 in the passageway 14 to be increased or decreased.

In order to provide an appropriate concentration, or intensity, of UV-C light, the inner surface 21 of the passageway comprises a reflective material. One such reflective material is aluminum, preferably polished aluminum. Other reflective material include silver, tin, or a reflective coating. Although not required, it is preferred that the entire inner surface 21 of the passageway 14 includes a reflective material. While not intending to be bound to any particular theory, it is believed that the reflective material will increase the intensity and concentration of UV-C light as the UV-C light travels from the source through the passageway 14 and out of the opening 18 in the passageway 14 and the opening 11 in the housing 12.

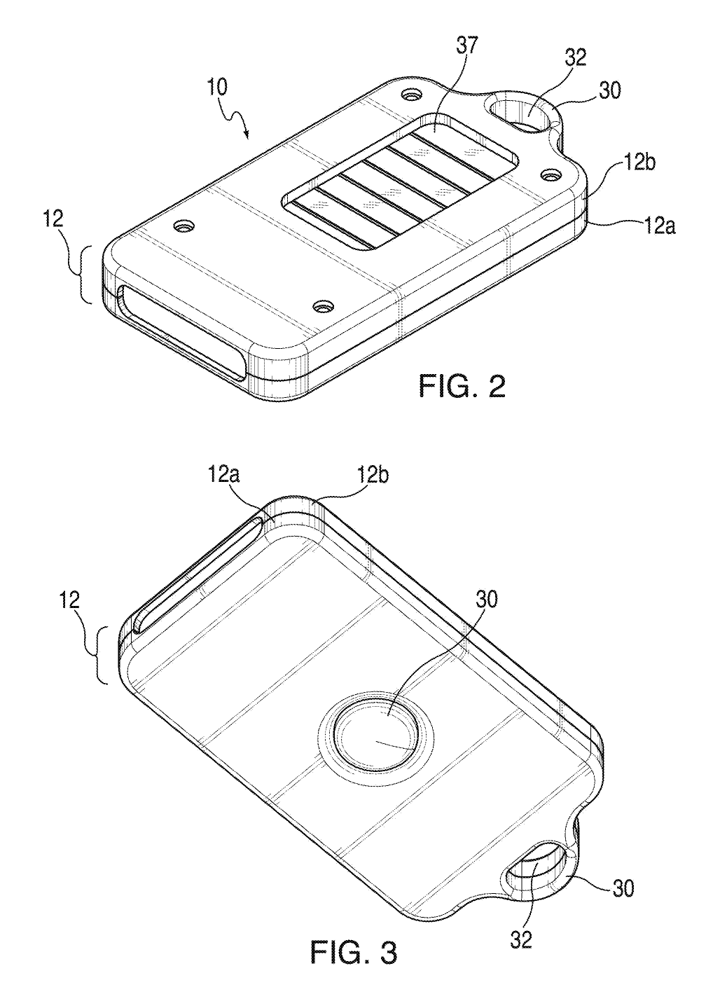

The device 10 also preferably includes an actuating element 28, such as a button 30, to turn the device 10 on and off Additionally, the device 10 includes a power source, such as a lithium ion battery 33 or other such battery, and a circuit board 35 with electronic components. In at least one embodiment, it is preferred that the device includes a solar panel 37, so that the battery 33 may be charged by solar energy when the device 10 is not in use. See, FIG. 2. Although not depicted as such, if different radiation emitting devices 22 (such as different LEDs) are utilized, it is contemplated that multiple actuating elements are used—for example a button or switch for a UV-C LED and a button or switch for a UV-A LED.

Turning to FIGS. 2 and 3, it is further contemplated that the housing 12 includes an extension 30 (or flange), extending away from the housing 12. An aperture 32 is disposed within the extension 30. The aperture 32 is sized to receive a key ring or other similar member, such as a d-ring, clip, chain, rope, lanyard, or any other such device.

It is contemplated that that a device according to one or more embodiments of the present invention will comprise a key fob sized device that measures 3.0 (±0.5) inches by 1.0 (±0.5) inch with a thickness of 0.375 of an inch (±0.25). For example, the device 10 shown in FIGS. 3 to 5 may be a key fob sized device. It is also contemplated that a device according to one or more embodiments of the present invention can be sized and configured to house a cellular phone. As such, the device may be larger and can form a case for the cellular phone. In one example, the devices may measure 2.5 inches by 5 inches with a thickness of approximately 1.0 inch. As will be appreciated, the size of a device which is configured to house a cellular phone can vary greatly depending on the size of a specific cellular phone.

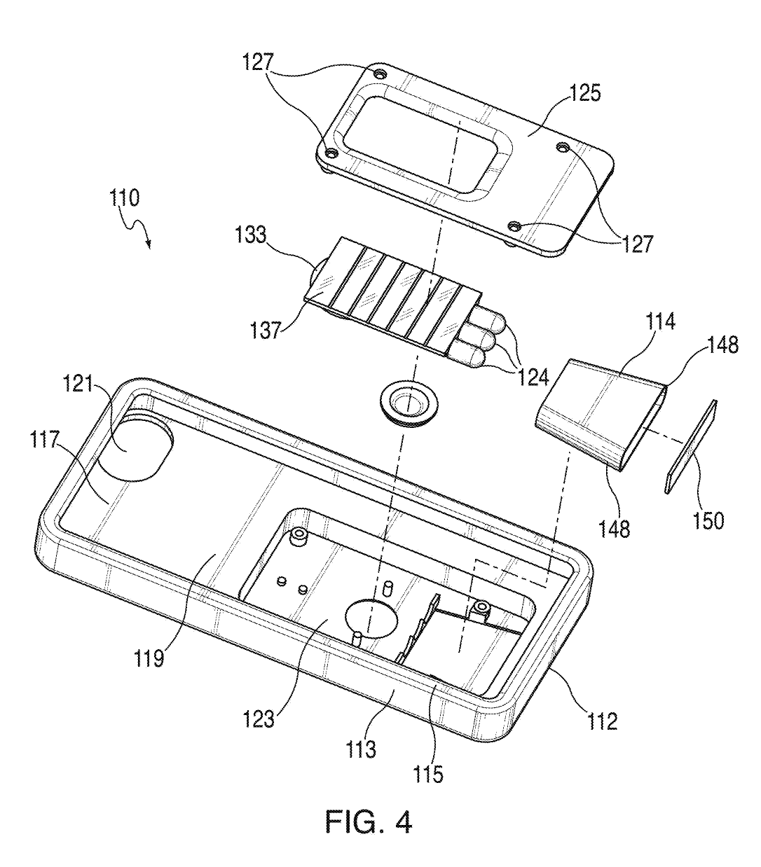

Turning to FIGS. 4 to 6, another device 110 according to one or more embodiments of the present invention is shown in which the housing 112 includes a side wall 113 and a top flange 115 which together with a back panel 117 form a cavity 119. The cavity 119 in the housing 112 is configured and sized to receive a mobile phone (not shown). It is contemplated that the top flange 115 of the housing 112 is comprised of rubber or another similarly flexible material to allow for the mobile phone to be easily inserted into or removed from the cavity 119 yet held in place when within the cavity 119. The housing 112 may also include one or more apertures 121 to allow for various buttons and equipment (camera, speaker, etc.) on the mobile phone to be utilized while the phone is contained within the housing 112.

As shown in FIG. 4, the back panel 119 of the housing 112 includes a recess 123 for the circuit board (not shown), solar panel 137, battery 133, LEDs 124 and other electronic components. Although not depicted as such, it is also contemplated that the power source for the device 110 may comprise the battery of the cellular phone.

Returning to FIG. 4, the passageway 114 may be removably contained within the recess 123 as well. A cover plate 125 may hold the various components of the device 110 within the recess 123 and be secured via fasteners, such as screws 127 or the like.

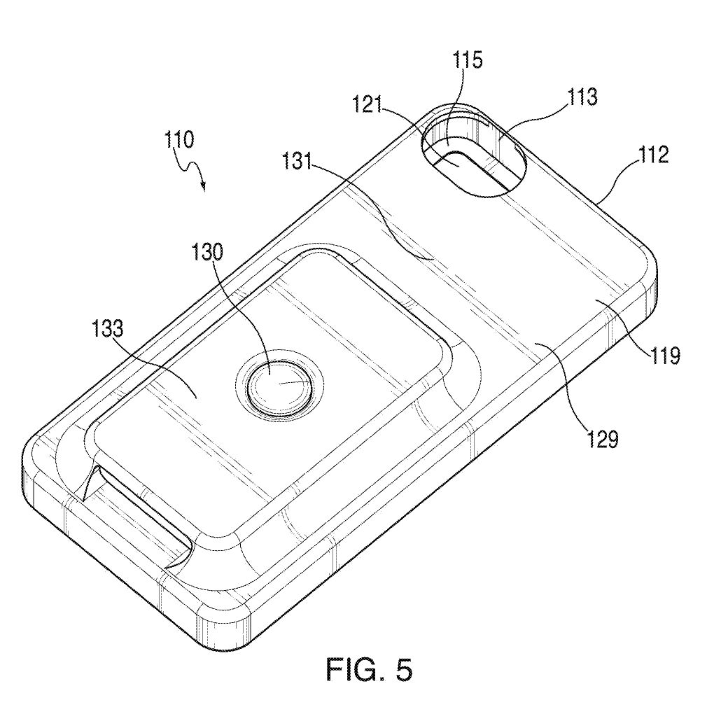

With reference to FIG. 5, an outer surface 129 of the back panel 119 of the housing 112 will include a first planar portion 131, corresponding to the cavity 119, and a second planar portion 133 corresponding to the recess 123. The second planar portion 123 will be raised in relation to the first planar portion 131. A button 130 may be disposed in the second planar portion 133 of the back panel 119 of the housing 112 to allow for the device 110 to be turned on and off.

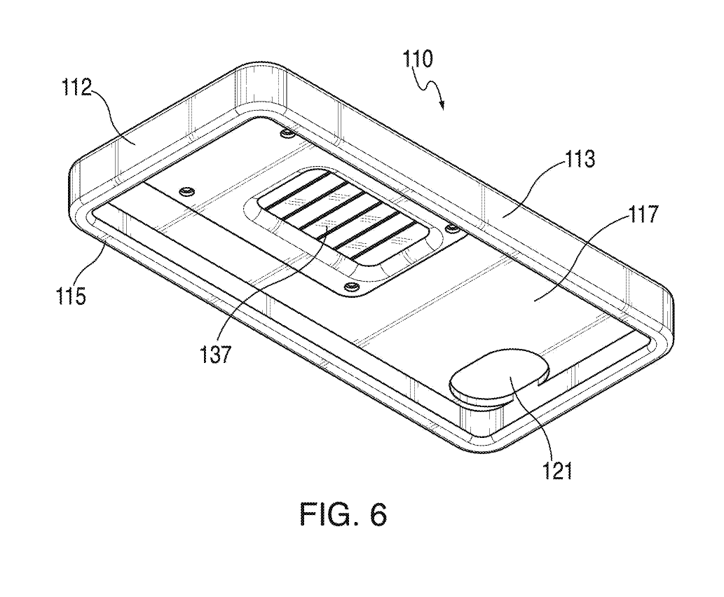

As shown in FIG. 6, when the mobile phone is removed, the solar panel 137 may be exposed, allowing the device 110 to be charged when not in use.

Returning to FIG. 4, in this embodiment, the device 110 also includes a lens 150 that is disposed proximate the second opening of the passageway, for example in the opening in the housing. See, FIG. 5. The lens can be used to focus the beam of light at a desired distance from the device 110, for example twelve inches. Additionally, the lens 150 may also protect the internal components of the device 110. The remaining portions of this embodiment may be similar to the device 10 shown in FIGS. 1 to 3, and those portions of the above description are incorporated herein.

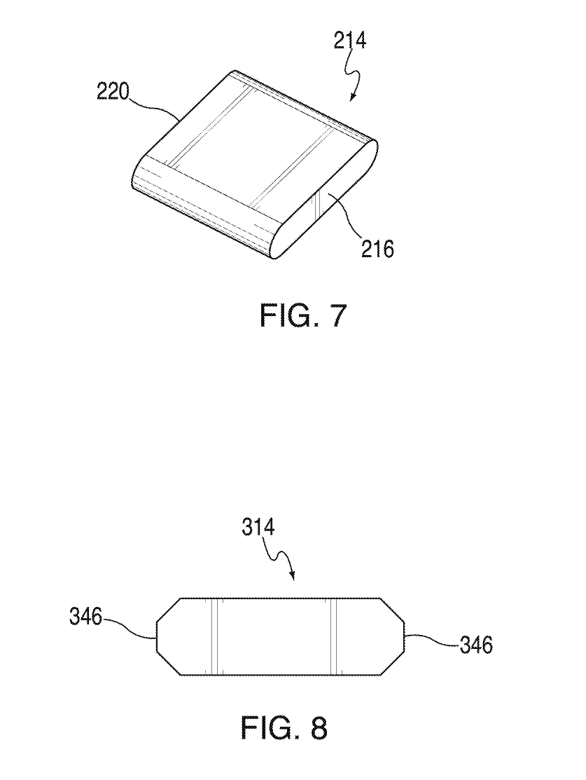

Turning to FIG. 7, a passageway 214 is shown in which the first end 216 and the second end 220 of the passageway 214 have the same approximate width. Such a passageway 214 could be used in any of the embodiments according to the present invention.

Additionally, as shown in FIG. 8, it is also contemplated that a passageway 314 may comprise an angular surface, that is, the passageway 314 may include angled side portions 346. As depicted in the embodiments of FIGS. 1 and 4, the passageways 14, 114 comprise a smooth surface with include curved side portions 48, 148, respectively. It is contemplated that the passageway 314 could be used in any of the embodiments according to the present invention.

Although not depicted as such, the devices according to various embodiments may include various safety or security measures or devices such as a “dead man's” switch, primary and secondary switches, altitude sensors, accelerometers.

The light (or other radiation) that is emitted from the devices according to the various embodiments of the present invention can be used to clean objects by utilizing the beam of the emitted light to destroy bacteria or other microorganisms. The various devices are easily portable and can be used in a wide variety of applications.

As is apparent from the foregoing specification, the invention is susceptible of being embodied with various alterations and modifications which may differ particularly from those that have been described in the preceding specification and description. It should be understood that I wish to embody within the scope of the patent warranted hereon all such modifications as reasonably and properly come within the scope of my contribution to the art.

1. A handheld bactericidal device comprising:
a housing;
an internal passageway disposed inside of the housing, the internal passage way comprising an opening at a first end and terminating at a second end within the housing; and,
at least one LED disposed within the internal passageway proximate the second end of the internal passageway and capable of emitting radiation capable of destroying DNA of microorganisms, wherein all radiation that exits the opening at the first end of the internal passageway of the housing is emitted from the at least one LED; and,
wherein an inner surface of the internal passageway comprises a reflective material.

2. The handheld device of claim 1, wherein the first end of the internal passageway is wider than the second end of the internal passageway.

3. The handheld device of claim 1, wherein all of the inner surface of the internal passageway comprises the reflective material.

4. The handheld device of claim 3, wherein the reflective material comprises polished aluminum.

5. (canceled)

6. The handheld device of claim 1 further comprising at least three LEDs capable of emitting UV-C light.

7. The handheld device of claim 1 wherein the housing further comprises an extension extending away from the housing and an aperture disposed in the extension.

8. The handheld device of claim 1 wherein the housing includes a cavity and the cavity is configured to contain a cellular telephone.

9. The handheld device of claim 1 further comprising:
a solar panel disposed in the housing.

10. The handheld device of claim 1 wherein the device measures about 3.0 inches by about 1.0 inches and has a thickness of about 0.375 inches.

11. The handheld device of claim 1 wherein when viewed from a front the internal passageway comprises side portions that are smooth.

12. The handheld device of claim 1 wherein when viewed from a front the internal passageway comprises side portions that are angled.

13. The handheld device of claim 1 wherein the first end of the internal passageway comprises a lens.

14. A bactericidal device comprising:
a housing comprising a back panel with a side wall extending away from the back panel to form a cavity, the side wall including a top flange.
an internal passageway disposed in the cavity of the housing, the internal passageway comprising an opening at a first end and terminating at a second end within the housing, the opening disposed in the side wall of the housing;
at least one LED capable of emitting UV-C light disposed within the internal passageway proximate the second end, wherein all radiation that exits the opening at the first end of the internal passageway of the housing is emitted from the at least one LED; and,
a solar panel disposed in the housing.

15. The device of claim 14 wherein the opening comprises a lens.

16. The device of claim 14 wherein the cavity is configured to receive a cellular telephone.

17. The device of claim 14 wherein the housing further comprises an extension extending away from the housing and an aperture disposed in the extension.

18. The device of claim 14 further comprising:
an actuating element in the housing.

19. The device of claim 18 wherein the actuating element comprises a button.

20. The device of claim 14 further comprising:
a power source disposed within the housing.

21. The device of claim 14 wherein the solar panel is disposed within the cavity of the housing.
